(19) United States
(12) Patent Application Publication
(10) Pub. No.: US 2020/0330008 A1
(43) Pub. Date: Oct. 22, 2020

(54) SKIN PATCH FOR DIAGNOSIS

(71) Applicant: BRIGHTER AB, Kista (SE)

(72) Inventor: Truls SJÖSTEDT, Täby (SE)

(21) Appl. No.: 16/956,728

(22) PCT Filed: Dec. 13, 2018
(86) PCT No.: PCT/EP2018/084763

§ 371 Date: Jun. 22, 2020

(30) Foreign Application Priority Data
Dec. 22, 2017 (SE) 1751628-7

(51) Int. Cl. A61B 5/145; A61B 5/1491; A61B 5/00

(52) U.S. Cl. CPC A61B 5/14514; A61B 5/14532; A61B 2560/04; A61B 5/685; A61B 5/688; A61B 5/1491

(57) ABSTRACT

There is provided a medical device (1) comprising a skin attachment surface (3) and an evaporation layer (9), and at least one hollow microneedle (4) extending from the skin attachment surface (3), the device (1) further comprising a flow channel (6), an analysis unit (8)for analysing a body fluid, and an evaporation layer (9), where the flow channel (6) is arranged to transport fluid from the microneedle (4) to the analysis unit (8) and from the analysis unit (8) to the evaporation layer (9), such that the flow channel (6) can release fluid into the evaporation layer (9).

### FIELD OF THE INVENTION

This invention relates to a medical device for attachment to the skin, the device comprising at least one hollow microneedle for measuring an analyte, for example glucose, in a fluid, for example interstitial fluid.

### BACKGROUND

Diabetes patients need to measure the glucose concentration in the blood on a regular basis, sometimes several times per day. Today, this is usually done by the patient piercing his or her own skin to produce a drop of blood which is then analysed. The drop of blood is collected by a glucose measuring stick that is inserted into a portable glucose measurement device. This procedure is carried out every time the blood glucose is measured. This is somewhat cumbersome as the patient needs to carry the device and test sticks and has to remember to carry out the test, and also has to puncture his or her own skin every time blood glucose is measured. This has the effects that some patients are reluctant to measure their blood glucose as often as they should. This can be very dangerous for the patient.

Abbot FreeStyle Libre provides one solution to this problem as it provides a skin patch with a needle that is inserted into the skin, which provides continuous blood glucose measurement. The FreeStyle Libre patch analyses blood glucose and can transfer measurement wirelessly to a portable device. However, this product causes some discomfort due to the length of the needle which is about 5 mm long.

Besides blood glucose, there are many different analytes that would benefit from continuous measurement, for example hormones or other signalling molecules, toxins, indicators of infection, etc.

WO90333898 discloses a device for sampling a fluid from a user where the fluid is collected in a closed chamber, with a limited space, making the device unsuitable for collecting large amounts of fluid.

Ventrelli et al, Adv Helthcare Mater. 2015, DOI: 10.1002/adhm.201500450 describes various arrays of microneedles where analysis takes place inside the microneedle. This has the disadvantage that the analysis electrode must be miniaturized which makes it difficult to manufacture.

Thus, there is a need for a more comfortable device for measurement of an analyte from the body of a patient, which can be used for an extended period of time.

### SUMMARY OF INVENTION

In a first aspect of the invention there is provided a medical device comprising a skin attachment surface and an absorption layer, which preferably is an evaporation layer, and at least one hollow microneedle extending from the skin attachment surface, the device further comprising a flow channel, an analysis unit for analysing a body fluid, and an evaporation layer, where the flow channel is arranged to transport fluid from the microneedle to the analysis unit and from the analysis unit to the evaporation layer, such that the flow channel can release fluid into the evaporation layer.

One advantage with his device is that the evaporation layer makes the device able to handle large amounts of fluid. Therefore, the device can be used over a long time. Furthermore, the analysis unit does not have to be miniaturized to a great extent.

The device may comprise a pump unit that transports fluid from the microneedle to the evaporation layer. The pump assists in creating a flow from the microneedle to the evaporation layer.

In a different embodiment the transport of fluid through the medical device is be driven by the evaporation layer. The flow may then be initiated by a single-use pump.

The device preferably comprises a main body, where the analysis unit is comprised in the main body. The at least one microneedle extends from the main body. Thus, the analysis unit is not comprised in the microneedle. This has the advantage of using the space in the main body for the analysis unit rather than having a part of the analysis unit in the hollow microneedle.

The evaporation rate from the evaporation layer may be the same or higher than the flow rate in the flow channel, in particular when the flow in the device is caused by evaporation.

The medical device may comprise at least one heat-generating electronic unit were the evaporation layer is arranged in contact with the at least one heat-generating electronic unit. This has the advantage of increasing evaporation from the evaporation layer. The device may comprise a heat conducting element that is arranged between the attachment surface and the evaporation layer. This has the advantage of conducting heat from the body of the user, increasing evaporation from the evaporation layer. The heat conducting element may be arranged between the evaporation layer and a heat generating electronic unit.

The heat-generating electronic unit or the heat conducting element may be provided with at least one surface area increasing element, such as at least one fin.

The evaporation layer has antimicrobial activity for preventing proliferation of for example bacteria. The fluid handling of the evaporation layer may be mainly based on absorption and retention of the fluid where evaporation plays a less significant part.

In selected embodiments, the evaporation layer can be removed from the medical device and be replaced with another evaporation layer. In one embodiment the device comprises a skin attachment layer which comprises the attachment layer where the skin attachment layer can be removed from the device and replaced with another skin attachment layer. The skin attachment layer may comprise the at least one microneedle. Thus, in one embodiment the skin attachment layer and the an the at least one microneedle can be removed from the medical device as one unit and replaced with another attachment layer. This enables reuse- of expensive parts of the device. Thus, the evaporation layer and/or skin attachment layer may be replaceable.

In a second aspect of the invention there is provided, an absorption layer which preferably is an evaporation layer for attaching to a medical device with a removable evaporation layer.

In a third aspect of the invention there is provided a skin attachment layer for attaching to a medical device with a removable skin attachment layer. The skin attachment layer may comprise at least one microneedle.

In a fourth aspect of the invention there is provided a method of analysing a body fluid comprising the steps of attaching a device as above to the skin of a user, collecting body fluid from the user with use of the at least one microneedle, using the analysis unit to analyse the body fluid, and allowing at least a part of the fluid to be absorbed by the evaporation layer, and allowing at least a part of the fluid to evaporate from the evaporation layer.

### BRIEF DESCRIPTION OF DRAWINGS

The accompanying drawings form a part of the specification and schematically illustrate preferred embodiments of the invention, and serve to illustrate the principles of the invention.

FIG. 1 is a schematic section side view of a medical device attached to the skin of a user.

FIG. 2 is a schematic section side view of an array of hollow microneedles.

FIG. 3 is a schematic diagram of various electronic units in a medical device.

FIG. 4 is a schematic section side view of a medical device.

FIG. 5 is a schematic section side view of a medical device.

FIG. 6 is a schematic perspective view of a medical device.

FIG. 7 is a schematic section side view of the medical device of FIG. 6

FIG. 8 is a schematic section side view of a medical device comprising an elastomer.

FIGS. 9 to 12, 13a and 14 are schematic side views of a medical device.

FIG. 13b is a schematic section side view of a detachable evaporation layer.

FIG. 13c is a schematic section side view of a reusable part.

FIG. 13d is a schematic section side view of a detachable attachment layer.

FIG. 14 shows a medical device attached to the skin of a patient.

### DETAILED DESCRIPTION

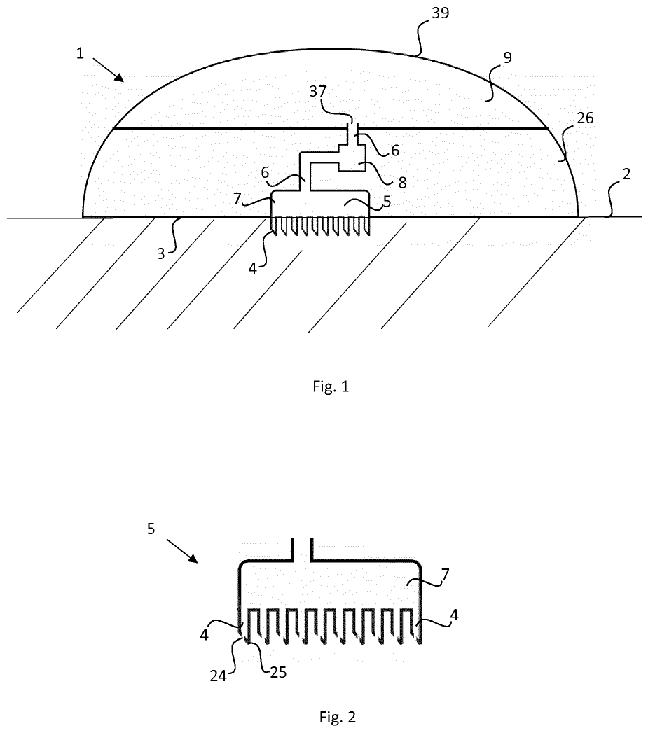

With reference to FIG. 1, the medical device 1 is intended for attachment to the skin 2 and to analyse a body fluid. The body fluid may be for example blood or interstitial fluid, where interstitial fluid is preferred. Suitable locations for attaching the device 1 may include the arm, for example the upper arm or the thigh.

The medical device 1 preferably has the shape of a patch or a flat housing. The device has at least one flat surface that is an attachment surface 3 for attaching the device to the skin 2 of a user. The at least one microneedle 4 protrudes from the attachment surface 3. The device 1 is intended to be attached to the skin 2 so that the at least one microneedle 4 is inserted into the body of a user. Thereby the microneedle 4 penetrates the outer surface of the skin 2.

The body fluid is extracted from the body with the use of at the least one hollow microneedle 4. Preferably there is an array 5 comprising a plurality of microneedles 4. When there is an array 5 the microneedles 4 are preferably arranged approximately in the same direction. The direction of the microneedle 4 may be approximately 90° (perpendicular) to the skin attachment surface 3, or may be slightly slanted in relation to the skin attachment surface 3. With reference to FIG. 2, which shows an array 5 of a plurality of microneedles 4, the at least one microneedle 4 is hollow and has at least one opening 24 close to the tip 25 or at tip 25 for receiving body fluid. The length of the microneedle 4 is preferably as short as possible in order to minimize discomfort (for example pain) of the user but should still be long enough to sample the body fluid of interest in a reliable manner. Longer microneedles 4 may be required for the sampling of blood, and sampling of interstitial fluid may therefore be preferred. The length of the microneedles may be from 50 μm to 3000 μm, preferably from 100 μm to 2000 μm, more preferably from 200 μm to 1500 μm. The internal diameter of the hollow microneedle may be for example in the range of from 15 μm to 300 μm. The microneedle 4 is preferably made of metal, silicon, or a polymer material, or a ceramic. Useful materials include platinum, titanium, iron, gold, nickel, copper, gold, or alloys of these metals. Stainless steel is also a useful material. U.S. Pat. No. 9,033,898 and references therein, and Ventrelli et al, Adv Helthcare Mater. 2015, DOI: 10.1002/adhm.201500450 provides information about suitable microneedles, arrays and their production.

The array 5 may have any suitable number of microneedles 4. For example, 1, 2, 3 or more, such as 10 or more, such as 100 or more, for example up to 500 microneedles 4. When there is a plurality of microneedles 4 they may be arranged along a line or as a matrix, for example in a square, or in a circle.

Because of the elasticity of the skin 2 an applicator may have to be used to insert the at least microneedle 4 or the plurality of microneedles 4 into the skin. An attachment speed of 1 m/s to 20 m/s may be useful. The applicator may be able to generate an acceleration of from 4 m/s² to 100 000 m/s².

Returning to FIG. 1, the microneedle 4 or microneedles 4 is connected to a flow channel 6 such that fluid collected in the microneedle 4 can flow into the flow channel 6. An array of microneedles 5 or all microneedles 4 of the device 1 may be connected to the same flow channel 6 with a system of branches 7.

Bodily fluid flows from the body of the user into the opening 24 of the at least one microneedle 4 and from the microneedle 4 into the flow channel 6. The fluid then passes by or through the analysis unit 8 where at least a part of the fluid is analysed. The flow channel 6 continues on the other side of analysis unit 8. The fluid then flows out into the storage layer which preferably is an evaporation layer 9 where the water component of the fluid is absorbed and then evaporates. Alternatively, the absorption layer may be a storage layer (see below). Analysis unit 8 may be incorporated into a part of flow channel 6. For example, analysis unit 8 may be an electrode that is arranged in the flow channel 6.

Flow through the device 1 may be at least partially achieved by evaporation from absorption layer (which may be evaporation layer 9) or absorption of fluid by absorption layer (which may be evaporation layer 9) or caused by a pump 10 (FIG. 10), or combinations thereof. Flow may be caused by capillary action and/or wicking action of the absorption layer (which may be evaporation layer 9) and/or evaporation, without the aid of a pump 10. Thus, in some embodiments flow through flow channel in device may occur without the aid of a pump 10. In some embodiments the pump 10 is be used to initiate flow through the device 1 and then cease to operate.

The flow channel 6 may come in a pre-wetted state such that it is delivered with some fluid (for example water) in the channel 6 to initiate evaporation and flow through the flow channel 6. However, capillary action may be enough to provide the initial flow. Capillary action is best achieved with a relatively thin flow channel.

The pump 10 may be such that it is used only to initiate flow from the microneedle 4 to the absorption layer, so that the absorption layer can drive flow once the flow channel 6 is filled. This may be useful if capillary flow is not enough to initiate flow. Then the pump 10 may preferably be a single use pump, for example a pre-packaged vacuum chamber, or a chamber that changes its shape with the aid of for example an electromagnetic force or an electric current. The task of the single use pump 10 is to fill the flow chamber 6 from the microneedle 4 to the evaporation layer 9 with bodily fluid. The pump 10 may be such that it ceases to operate once flow through the device 1 has been established. The single use pump may be activated by attaching the device 1 on the skin 2. For example, the single use pump may be activated by body heat. Alternatively, the attachment surface 3 may have a bulge or button that is displaced when the user attaches the device to the skin, and the button may cause a suction in the flow channel 6. As a third option, pumping action may be caused by the user pressing a button.

The flow channel 6 is preferably a microfluidic channel. The diameter of the flow channel 6 is typically less than 1 mm, more preferably less than 500 microns and in some embodiments less than 50 microns, and in some embodiments less than 1 micron.

The flow channel 6 may be constituted by parts of housing 18 or inner housing 28 or may be constituted by separate piping. The material of the piping of the flow channel 6 may for example be a polymer material, but for example metal may also be used.

The flow rate of the device 1 may be selected so that a reliable analysis, typically analyte concentration measurement, is obtained, while keeping the device 1 and, in particular, microneedle 4 as small as possible. Any useful schedule for flow and analysis may be used. Flow rates through the device 1 of between 1 nl/hour to 300 μl/hour may be used, where 100 nl to 30 μl/hour is preferred. It may be necessary to use a pump 10 to obtain the higher flow rates. Flow may be more or less constant or may vary over time. For example, flow may be increased at certain time points or may cease or almost cease at other time points. Flow in the flow channel 6 may be controlled with the use of pump 10, or valves. Valves and pump 10 may be controlled by processor 11.

The optional pump 10 may be any type of mechanism that creates suction or pressure. In one embodiment the pump 10 is powered by electricity. The pump 10 may for example be a piezoelectric or an electromechanical device. The pump 10 may alternatively be a pre-packaged vacuum chamber, or another device that creates a vacuum, for example with the help of memory foam, or chamber that changes its shape over time, thereby creating suction or pressure. The pump 10 may be placed between the microneedle 4 and the analysis unit 8 or between the analysis unit 8 and the evaporation layer 9.

The analysis unit 8 is able to detect at least one property of the bodily fluid. The property may be the absence or presence or concentration of an analyte. Any useful analyte may be analysed by analysis unit 8. Examples of analytes include: glucose, pH, electrolytes, liver enzyme values, biomarkers, c-reactive protein, immunoglobulin, pharmaceuticals or their breakdown products, hormones or other signalling molecules, peptide or peptide fragments, toxins, metabolic products, substances from pathogens such as bacterial or viral toxins or proteins, and lipids such as cholesterol. The biomarker may be an endogenous protein or a pathogen protein, for example a virus, bacterial or parasite protein.

Any useful chemistry or method can be used to analyse the analyte. For example, electric potential, spectroscopy, fluorescence, immunoassays, light scattering, surface plasmon resonance, binding of a specific reagent such as an antibody, or an enzyme activity, or combinations thereof may be used, as long it may be miniaturized to an extend to fit into device 1, in particular main body 26 of device 1 (see below).

Glucose is a preferred analyte. Thus, the device 1 is, in a preferred embodiment, adapted to analyse the level of glucose in the interstitial fluid or in the blood of a user. Methods of detecting the level of glucose are well known. Continuous measurement of glucose levels is suitably measured using conventional glucose oxidase chemistry using electrodes. Typically, three electrodes are used: a working electrode, a counter electrode and a reference electrode. Typically, the enzyme glucose oxidase is used for catalysing the generation of H₂O₂ at the working electrode. Glucose oxidase can for example be captured in a layer on the electrode. A surplus of O₂ is needed for this reaction to occur at the working electrode, and a mediator compound may be used for generating free electrons from H₂O₂, in order to decrease the need for O₂. Useful mediators include ferrocene derivatives, ferricyanide, conducting organic salts (particularly tetrathiafulvalene-tetracyanoquinodimethane, TTF-TCNQ), phenothiazine and phenoxazine compounds, or quinone compounds. Alternatively, glucose hydrogenase may also be used as the enzyme. Ventrelli et al, Adv Helthcare Mater. 2015, DOI: 10.1002/adhm.201500450 provides information about useful glucose sensors.

Analysis of the glucose levels of a user, in particular a diabetic patient, by analysing interstitial fluid, is a preferred embodiment. Glucose concentrations of the interstitial fluid closely reflects that of the glucose concentration in blood, but with a slight time lag.

Other types of analytes that can be measured with electrodes includes glutamate, ethanol, choline, cortisol or lactate. For example, electrodes of the type sold by Pinnacle Technology, Kans., USA, may be used. Preferably analysis unit 8 includes at least one electrode 8 for measuring the presence or concentration of an analyte.

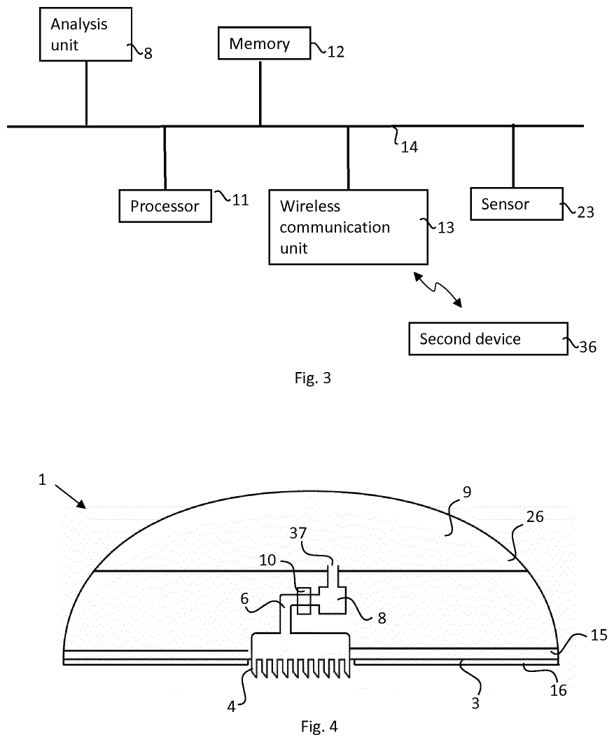

With reference to FIG. 3 the device 1 preferably comprises a processor 11 for collecting signals from the analysis unit 8 and a memory 12 for storing measurement values and software, and a wireless communication unit 13. The wireless communication unit 13 is preferably able to transfer information to a second device 36, for example a smartphone or other type of wireless device, or other type of device. Preferably sample data is be transferred together with a time point for analysis or sampling. Data transfer may occur automatically when the second device 36 is within range of the medical device 1. The wireless communication may be of the near field communication (NFC)-type for example Bluetooth®.

The device 1 preferably comprises a communications interface 14 for allowing communication between the various electronic components. There may be a power source such as a battery and wiring for powering one or more of analysis unit 8, pump 10, processor 11, memory 12, wireless communication unit 13 and communications interface 14 and a sensor 23, for example a flow sensor or a pressure sensor. The battery may be charged with the use of an induction coil, or a port, or may be disposable. As an alternative to a battery the device may have self-powered biofuel cell (BFC). Analysis unit 8, pump 10, processor 11, memory 12, wireless communication unit 13, communications interface 14, battery and sensor 23 are all referred to as “electronic units” 29 herein. The device 1 may comprise other electronic units such as, but not limited to: sensors, alarms, light emitting units, charging coils, valves, etc. These are also referred to as “electronic units” 29. The device 1 may have at least one electronic unit 29, in particularly an electronic unit 29 that generates some heat as a by-product if its operation. For example, a wireless communications unit 13 generates at least some heat during transmission, and processor 11 generates at least some heat when it is working.

Processor 11 and software stored in memory 12 control or receive data from the various electronic units 29 of device 1. Processor 11 and software may control flow of fluid trough device 1 by controlling pump 10 or a valve. Sampling or analysis may be done at any suitable interval and may be controlled by processor 11. Sampling or analysis may be carried out on at least every predetermined timer interval with may be for example at least every 24 hours, more preferably at least every 12 hours, at least every 6 hours, at least every 3 hours, at least every 2 hours, at least every 60 minutes, at least every 30 minutes, at least every 15 minutes, at least every 10 minutes, at least every 5 minutes, at least every minute, at least every 10 seconds or at least every second. Pump 10 and valves may operate in conjunction with the sample frequency. The data from analysis unit 8 may be stored in memory 12 together with a related timepoint such as time and date for sampling or analysis. When analysis is done in a fluid that flows more or less continuously past a sensor, for example an electrode, the time point for analysis may be more relevant. In some embodiments, fluid may be isolated before analysis and in those cases the time point for sampling may be more relevant.

Transfer of data to second device 36 may be done automatically with a predetermined schedule, which may be at least once per day, or at the convenience of the user. Transfer of data may be initiated by the user, for example by using second device 36 to trigger data transfer. The processor 11 may control the sampling, analysis and transfer of data with the use of wireless communication unit 13 to the second device 36. For example, the device 1 and the second device 36 may carry out a handshake before transfer of data to the second device 36. The second device 36 may be able to store the data. Second device 36 may also be able to display the data on a display. Second device 36 may also be able to transfer data to a cloud solution for storage, analysis and future reference.

The processor 11 and software may be configured to carry out one or more of the following: monitoring the flow rate trough device 1, controlling flow rate, controlling the pump 10 and/or valves, monitoring the correct function of the device 1, cause an alarm in case of malfunction or need of replacement, check of battery status, alarm in case of low flow, starting and stopping the device, resetting the device, wireless communication with second device 36, data analysis, data storage and transfer, data encryption, storage of an ID of device 1 and of second device 36.

Preferably the device 1 is adapted to be used for several days or weeks, such as at least 3 days, more preferably at least 5 days, more preferably at least 10 days and most preferably at least 20 days. Thus, the device 1 is able to withdraw bodily fluid and analyse it for such a period of time.

Evaporation Layer

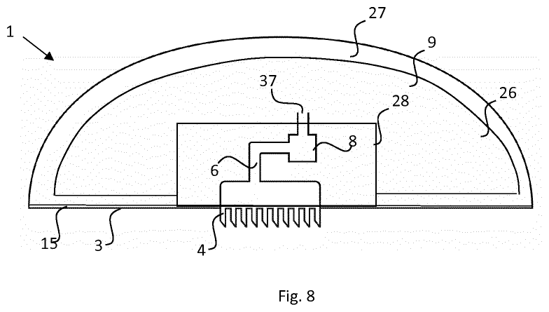

The device has an absorption layer. The absorption layer may be an evaporation layer 9 or, alternatively, a storage layer. The fluid handling capacity of the storage layer is completely or almost completely based on absorption of the fluid, where evaporation is negligible. All embodiments of this invention with an evaporation layer, can, whenever possible, also be used with a storage layer instead of an evaporation layer 9. An advantage with using a storage layer is that the absorbent material may be encapsulated, enabling the user to use the device 1 in the water, for example while swimming. An embodiment with an elastomer layer 27 may be particularly useful for this, see FIG. 8 (see below). However, in a preferred embodiment, the device has an evaporation layer 9 and the fluid handling is based at least in part on evaporation. Thus, the evaporation layer 9 may assist in the evaporation of the fluid.

The fluid handling capacity of the device 1 may be such that the fluid is handled by device 1 at least as fast as the flow rate in the device.

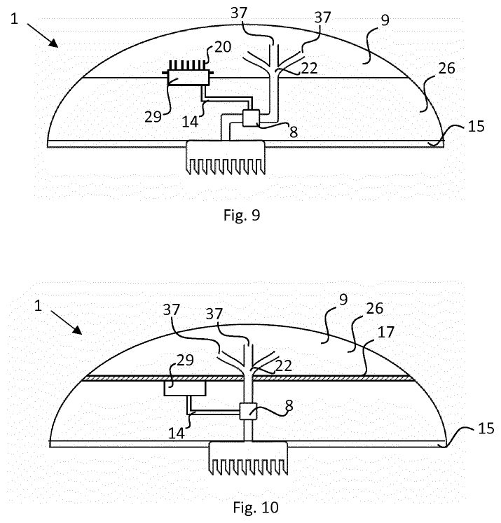

Returning to FIG. 1, the flow channel 6 disgorges fluid into the evaporation layer 9 through opening 37. The flow channel 6 may branch into two or more branches 22 (shown in FIGS. 9 and 10) that empties into the evaporation layer 9 trough opening 37. Therefore, there may be more than one opening 37. The purpose of branches 22 is to spread the fluid in the evaporation layer 9. The evaporation layer 9 is suitable to receive the fluid from the flow channel 6. The fluid is preferable water-based such as interstitial fluid or blood. The purpose of the evaporation layer 9 is at least one of the following: a) handle the fluid that is collected by the microneedle 4 by absorbing the fluid and then causing the fluid to evaporate (where not necessarily all fluid is evaporated) and b) enhancing or causing flow through device 1 by absorption and by evaporation or of the fluid. Preferably both a) and b) are achieved.

The evaporation layer 9 is able to absorb and/or allow fluid to evaporate. The evaporation layer 9 suitable has a high fluid handling capacity. As used herein “fluid handling capacity” means the combined ability to take up (absorb) moisture and to allow it to evaporate into the environment (the ambient air). The fluid handling capacity of the evaporation layer may be, for a layer with even thickness, at least about 1 g/m²/24 h, more preferably at least 10 g/m²/24 h, more preferably at least 500 g/m²/24 h, more preferably at least 1000 g/m²/24 h more preferably 2500 g/m²/24 hours or at least about 3500 g/m²/24 hours.

The fluid handling capacity of the evaporation layer 9 may be mainly based on absorption and retention (storage) of the fluid where evaporation plays a less significant part or may be based mainly on evaporation where most of the fluid is evaporated. Over time, storage may be a more important component in the initial phase of use of the evaporation layer 9, and evaporation may be a more important part as the evaporation layer reaches saturation. At steady state, all fluid handling capacity may be provided by evaporation. In one embodiment, at least 50%, more preferably 60%, even more preferably 70%, even more preferably 80%, even more preferably 90%, even more preferably 95% and most preferably at least 99% of the total fluid handling capacity of the evaporation layer during its time of use is provided by evaporation.

Moisture vapor transmission rate (MVTR) is a measure of the passage of water vapor through a substance. In one embodiment the evaporation layer 9 has a high MVTR. The MVTR of the device 1 may be such that the fluid evaporates with the same rate, or faster, than the flow rate of fluid in the device 1, such that all the fluid that reaches the evaporation layer 9 evaporates over time. The evaporation layer 9 may, when it has an even thickness, have a MVTR of at least 1 g/m²/24 hours, more preferably 300 g/m²/24 hours, more preferably at least 500 g/m²/24 hours, more preferably at least 1000 g/m²/24 hours, and most preferably at least 1200 g/m²/24 hours. A high MVTR is useful because it prevents antimicrobial growth and prevents formation of moisture, thereby providing more comfort to the user. Methods for determining fluid handling and MVTR are described in WO2013071007.

The evaporation layer 9 may be at least partially exposed to the surrounding air, which facilitates evaporation.

The evaporation layer 9 may comprise a material with a large surface area, such as a fluff layer, a foam layer, a porous layer or a sponge-like layer.

The evaporation layer 9 may comprise a cellulose fiber, such as cellulose fluff (fluff pulp). Examples of useful materials include BASF Luquafleece®, Texsus Absorflex® and similar products. Examples of absorbent materials are also provided in WO9620667 and WO200115649.

The evaporation layer 9 may comprise or consist of a hydrogel-forming absorbent polymer. Super absorbent polymers include crosslinked acrylate polymers, crosslinked products of vinyl alcohol-acrylate copolymer, crosslinked products of polyvinyl alcohols grafted with maleic anhydride and carboxymethylcellulose. BASF superabsorbent polymers, including the Medi Gel® and Artic Gel® brands of polyacrylate are useful.

The evaporation layer 9 may comprise a cellulose fiber in combination with a hydrogel-forming absorbent polymer.

When the absorption layer is a storage layer it may comprise or consist of the same type of materials as the evaporation layer. In particular, a hydrogel-forming absorbent forming polymer may be used for the storage layer.

The shape of the evaporation layer 9 may preferably be chosen to have a large surface area. For example, it may have a more or less flat constitution. Preferably the thickness of the evaporation layer 9 is less than both the width and the length of the evaporation layer 9. It is preferred that the maximum thickness of the evaporation layer 9 is at most 50%, more preferably at most 30% of the maximum width of the device 1. When the device 1 is puck-shaped (FIGS. 6 and 7) the width is the diameter of the device 1.

The device may have an outer layer 39, which may be breathable outer layer 39, most preferably a thin breathable layer (FIGS. 6-7) outside the evaporation layer 9. The outer layer 39 may serve the purpose of protecting against water splash, contamination or mechanical wear. The outer layer 39 may preferably comprise a non-woven material or a film material. The material may be water-vapor permeable. The outer layer 39 may for example have small holes or pores for increasing breathability. The outer layer 39 may for example comprise or consist of polyurethane, elastomeric polyester or polyvinyl chloride. The outer layer may be of a Gore-Tex type, i.e. a material which prevents against water seepage, but which is still breathable.

With reference to FIG. 4 the device 1 may preferably comprise an adhesive skin attachment layer 15 that comprises the attachment surface 3. The adhesive attachment layer 15 comprises an adhesive compound or composition that makes the device 1 adhere to the outer surface of the skin 2, even if applied to a vertical body surface, and during movement. The adhesive of the adhesive attachment layer 15 may be an acrylate, including methacrylates and epoxy diacrylates. The adhesive may be a pressure sensitive adhesive. Henkel Duro-Tak® is a useful adhesive. The adhesive attachment layer 15 may also comprise an elastomeric compound. The adhesive attachment layer may also be comprised in a separate housing that is detachable from the rest of device 1.

The adhesive attachment layer 15 may have a release liner 16 on the skin-contacting side. The release liner 16 is removed just prior to application of the device 1 to the skin 2 of the user.

With reference to FIGS. 1, 4 and 5 to 13 and 15, the medical device 1 may have any suitable shape. The medical device preferably has a main body 26 comprising the attachment surface 3, where the at least one microneedle 4 protrudes from main body 26. The main body comprises the evaporation layer 9, attachment surface 3, the major parts of flow channel 6, analysis unit 8, attachment layer 15 and any electronic units 29. The main body 26 may also comprise the base of the microneedle 4, such that the microneedle 4 protrudes from a base integrated in the main body 26. Preferably device 1 consist of main body 26 and the at least one microneedle 4.

Preferably the main body 26 has a rounded shape such as puck-shaped, or shaped as a patch. The patch may be a soft patch. Preferably the analysis unit 8 is comprised in the main body 26 of the medical device 1. For example, inside the housing 18 or embedded in the elastomer 27 as described below. Thus, preferably, no part of the analysis unit 8, for example electrodes, are inside microneedle 4.

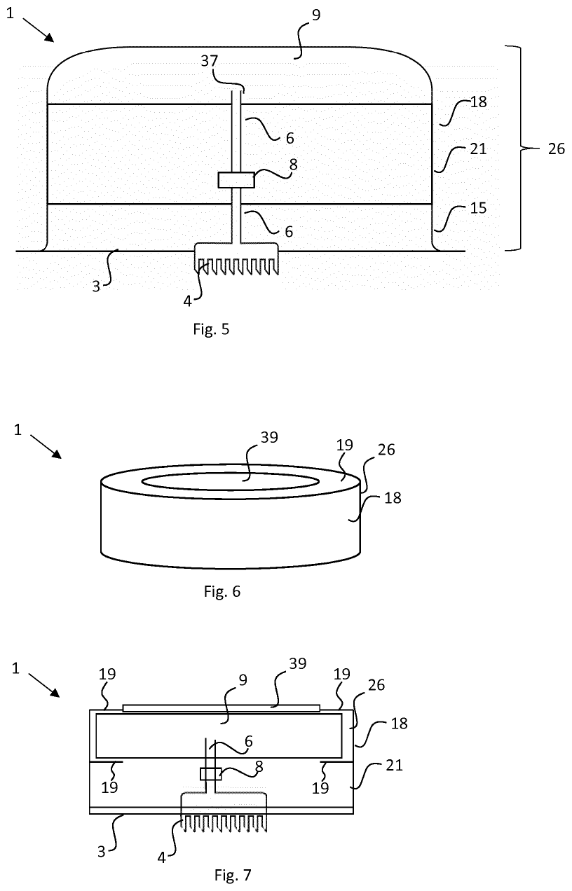

Analysis unit 8 and other components, such as other electronic components 29, may be arranged in a reusable part 21 (FIGS. 5, 7, 9 and 13) of the main body 26 of device 1, located between the evaporation layer 9 and the adhesive attachment layer 15.

The device 1 may, as seen in FIGS. 5, 6, 7, have an outer housing 18 with the evaporation layer 9 placed on top of the outer housing 18 or in an upper compartment of the housing 18. The housing 18 may be made of a stiff polymer material such as for example ABS, PET, PETG or polycarbonate. The evaporation layer 9 sits on top of the housing 18 or in an opening in the housing 18 and may be held in place with flanges 19 (FIG. 7).

The device 1 may comprise an elastomeric (rubber-like) material. The main body 26 of device may then be a soft patch. For example, the flow channel 6 and the base of the array 5 of microneedles 4 and at least one electronic unit 29, preferably the analysis unit 8, or all electronic units 29, may be contained in an elastomeric layer 27. With reference to FIG. 8, the elastomeric layer 27 may contain or surround the evaporation layer 9, in particular an evaporation layer 9 that absorbs a large amount of fluid such, as a hydrogel forming polymer. This embodiment may be particularly useful when a storage layer instead of an evaporation layer 9 is used. Preferably the elastomer layer 27 is non-permeable to water in the liquid phase so that the storage layer or evaporation layer 9 is not wettened when the user swims or takes a shower. Preferably a hydrogel-forming compound is used for the storage layer in the embodiment shown in FIG. 8. The flow channel 6 and the analysis unit 8 may be contained in an inner housing 28 that is embedded in the elastomeric layer 27 and the evaporation layer 9. Inner housing 28 may also comprise other electronic units 29. Inner housing 28 may also comprise the base for the array 5. Piping for flow channel 6 and inner housing 28 may be moulded in one piece of a polymer material.

The elastomeric layer 27 may be breathable in order to let water vapor through. For example, the elastomeric layer 27 may have pores. However, fluid handling may be almost completely based on absorption in evaporation layer 9 inside elastomeric layer 27. The elastomer layer may then have a low MVTR (low breathability), in order to protect layer 9 from moisture from the outside.

Examples of useful elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones.

Enhancement of Evaporation

Evaporation of fluid from the evaporation layer 9 may be enhanced by body heat from the user. Body head may be conducted or radiated from the user through the attachment layer 15 into the evaporation layer 9. Heating of evaporation layer 9 by the body of the user is facilitated when the thickness of the device 1 is low, making the evaporation layer 9 come close to the body. The maximum thickness of device 1 (measured from the attachment surface 3 to the top of the evaporation layer 9 or the outer layer 39) is preferably less than 20 mm, and even more preferably less than 10 mm and most preferably less than 5 mm.

Evaporation of fluid from the evaporation layer 9 may also be enhanced by heat from at least one electronic unit 29. At least one electronic unit 29 may be arranged so that it at least partially is arranged in contact with the evaporation layer 9 (FIG. 9). The electronic unit 29 may for example be selected from the group consisting of analysis unit 8, pump 10, processor 11, memory 12, wireless communication unit 13, communications interface 14, sensor 23, and battery.

The device 1 may comprise a heat conducting element 17. The heat conducting element 17 aid conduction and distribution of heat from the body of the user and from any electronic units 29 of the device 1 to the evaporation layer 9. The heat conducting element 17 may be for example a heat conducting layer, for example a metal film or foil as shown in FIG. 10.

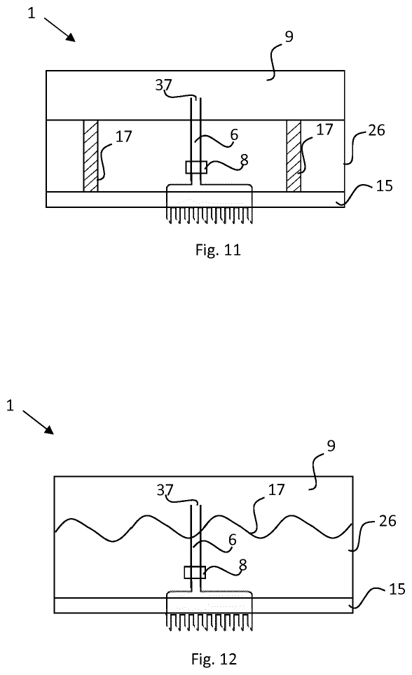

The heat conducting element 17 may be arranged between the attachment layer 15 and the evaporation layer 9. At least some part of the heat conduction element 17 is preferably in contact with the evaporation layer 9. The heat conducting element 17 may be arranged as shown in FIG. 10, above an electronic unit 29 but below the evaporation layer 9, thereby transferring heat from the electronic unit 29 to the evaporation layer 9 and thereby enhancing evaporation. Thus, the heat conducting element 17 may be arranged between the evaporation layer 9 and one component selected from analysis unit 8, pump 10, processor 11, memory 12, wireless communication unit 13, communications interface 14, sensor 23 and battery. The heat conducting element 17 may be a layer that is approximately parallel to the skin attachment surface 3 as shown in FIG. 10. However, the heat conducting element 17 may also be arranged as vertical metal studs or metal membranes or metal sheets that conduct heat from the body of the user to the evaporation layer (FIG. 11). The heat conduction element 17 then preferably stretches from close to the attachment surface towards the evaporation layer 9. Part of heat conducting element 17 may even be in contact with the outer surface of skin 2 of user when device 1 is attached to the skin 2. For example, the studs of Fig. may be in contact with the surface of the skin 2. In general heat conducting element 17 may be arranged to conduct heat in a direction that is perpendicular to the attachment surface 3 and towards the evaporation layer 9.

The heat conducting element 17 or an electronic unit 29 may comprise a heat conducting surface area increasing element such as heat conducting fins 20 or similar arrangement that increases the surface area for conducting heat to the evaporation layer 9. The fins 20 are preferably made of material with a high thermal conductivity, such as a metal. A part of the surface-area increasing arrangement, such as fins 20, may be embedded in the evaporation layer 9. The electronic unit 29 may have metal fins 20 attached to its outer surface, where the metal fins 20 are in close proximity or embedded in the evaporation layer 9 (FIG. 9). For example, the processor 11, the analysis unit 8 or the wireless communication unit 13 may be placed in close proximity to the evaporation layer 9 and have fins 20 that are embedded in the evaporation layer 9. As an alternative to fins 20, metal threads, metal wire or metal mesh, or similar may be used. As a further example, the heat conducting element 17 may be a corrugated metal foil, of which one side is embedded in the evaporation layer 9 (FIG. 12).

FIGS. 9 and 10 also shows communications interface 14 of device 1.

Antimicrobial Agent

The device 1, in particular the evaporation layer 9, may comprise an antimicrobial agent, in particular an antibacterial, antifungal or antiviral agent. Other parts that may comprise an antimicrobial agent are the at least one microneedle 4 and the inner surface of the flow channel 6, and analysis unit 8.

In one or more embodiments, the antibiotic agent is selected from the classes consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, alcohols, antibiotic polyethers, quinolones, antibiotic steroids, sulfonamides, tetracycline, dicarboxylic acids, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Examples of particularly useful antimicrobial agents may be parachlorometaxylenol; chlorhexidine and its salts such as chlorhexidine acetate and chlorhexidine gluconate; iodine; iodophors; poly-N-vinylpyrrolidone-iodophors; silver oxide, and silver and its salts, fucidic acid, sodium fucidate, retapamulin, mupirocin, oxytetracycline, polymyxin B, kanamycin, bacitracin, bacitracin zinc, neomycin, lactic acid, citric acid, and acetic acid.

Examples of anti-fungal agents which may be used in the present invention and which are known for their topical use are amorolfine, clotrimazole, miconazole, ketoconazole, ciclopirox, or terbinafine.

The antimicrobial agent is preferably a solid at room temperature, preferably a solid at a temperature at a temperature up to a 35° C. This temperature may be desirable in order to maximize evaporation of the body fluid from the evaporation layer 9.

Parts of the device 1 such as the at least one microneedle 4 or inner surface of flow channel 6 or the evaporation layer 9 may also comprise a surface treatment or coating that inhibits microbial growth. Example of such a coating or surface treatment are: arrangements that inhibits attachment of bacteria, agents that destroys the cell membrane of microbes, such as, for example, a surfactant or a membrane piercing protein, and the use of silver ions.

Replaceable Parts

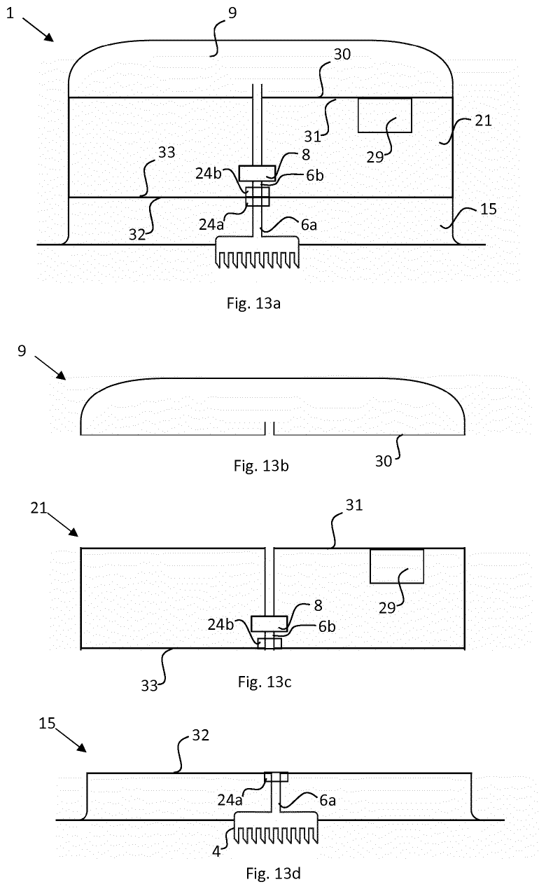

In certain embodiments (which can be combined with other embodiments) with reference to FIGS. 13a-d, at least one of the evaporation layer 9 and the adhesive attachment layer 15 (including microneedle 4) of the medical device 1 can be replaced. Thus, the evaporation layer 9 may have a lower surface 30 that is removably attached to the upper surface 31 of the rest of the device 1. The attachment layer 15 comprising attachment surface 3 may have an upper surface 32 which is removably attached to the lower surface 33 of the rest of the medical device 1, for example housing 18 or elastomeric layer 27.

In a preferred embodiment the evaporation layer 9 and the attachment surface 3 can be replaced while reusable part 21 comprising at least one electronic unit 29, in particular the analysis unit 8, is reusable. It is preferred that reusable part 21 comprises other electronic units 29, which may be expensive and thus can be reused, such as pump 10, processor 11, memory 12, wireless communications unit 13.

In one embodiment the battery is provided in one of the replacement parts 9 or 15. I this way the battery is changed when changing the evaporation layer 9 or the attachment layer 15. Electric supply cable between battery and electronic units 19 may then have a physical connector in much the same way as channel 6 (see below with reference to FIG. 13a).

The evaporation layer 9 (FIG. 13b) or the attachment layer 15 (FIG. 13d) or both may thus be reversibly attached to the rest of device 1, for example reusable part 21 (FIG. 13b), with for example an adhesive. The adhesive is preferably of a kind that allows attachment and detachment without breaking the reusable part 21. Attachment an also be achieved with for example micro Velcro or similar means.

Reusable part 21 may have any suitable design. For example, reusable part 21 may comprise outer housing 18. When the device has the embodiment of FIG. 8, the reusable part may comprise internal housing 28.

The detachable attachment 15 layer of FIGS. 13a and 13d may comprise a housing (separate from housing 18) or may comprise a body of an elastomeric material.

Preferably the replaceable attachment layer 15 comprises the microneedle 4, for example an array 5 of microneedles 4. The microneedle 4 or lower part of the flow channel 6a may become connected to the rest of the flow channel 6b by means of connectors 24a and 24b (FIGS. 13a, c, d). Preferably the connection between connectors 24a and 24b is leak proof, for example with the aid of a gasket or a press fit, or both. Connectors 24a and 24b may have quick lock, for or example the connectors 24a, 24b may snap together.

The flow channel 6 may end on the upper surface 31 of the reusable part 21 such that the flow channel 6 spills out into the evaporation layer 9 when the evaporation layer is attached to the reusable part 21. Alternatively, the replaceable evaporation layer may have a continuation of the flow channel (FIG. 13b) that is connected to the flow channel 6b with connectors similar to connectors 24a and 24b. This may be useful in particular when the flow channel has branches in the evaporation layer 9 (FIGS. 9 and 10).

Replacement of the skin attachment layer or the evaporation layer may be triggered by different events, for example at certain time points. For example, the evaporation layer or the skin attachment layer may be replaced within a predefined time limit, which may be for example 7 days or 14 days. Preferably replacement can be carried out by the user himself/herself. The device 1 may have alarms or chemical colour indicators that remind the user of replacement. For example, processor 11 may trigger a sound alarm after a predefined time point. The evaporation layer 9 may have be provided with a chemical colour change indicator that changes colour after being wettened for a certain time.

Further Considerations

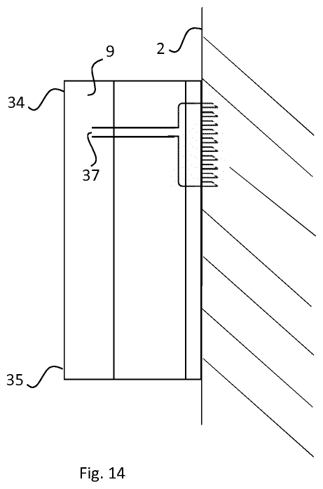

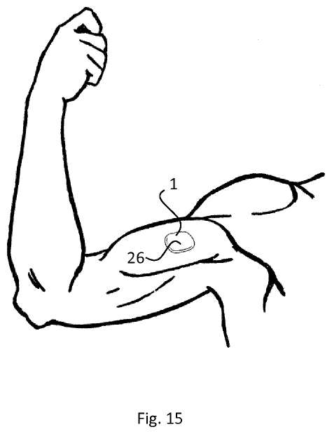

As seen in FIG. 14, the device 1 may have an upper end 34 and a lower end 35 and the flow channel 6 empties into the evaporation layer 9 closer to the upper end 34 than the lower end 35. When the device 1 is placed on a part of the skin 2 which is often a vertical or almost vertical surface, such as the side of the upper arm (at least when the user is not lying down), this arrangement will improve the distribution of liquid in the evaporation layer 9, as gravity will tend to make the fluid flow downwards from opening 37 of flow channel 6 into the evaporation layer 9. The flow channel may have branch 22, providing a plurality of openings 37.

In general, the main body 26 of device 1 may have any suitable shape. FIG. 15 shows a medical device 1 in the shape of a patch attached to the skin of the arm of a patient. The device 1 of FIG. 15 has a main body 26 which is the shape of a patch which has the shape of a square with rounded edges. The shape of the patch may be circular, oval or have any suitable shape. Preferably the main body 26 is as flat as possible. Preferably the edges of the main body 26 are rounded as seen in FIG. 15 and other figures in order not to snag to clothing, etc.

The shape and the size of device 1 and the attachment layer 15 should be selected so that the device 1 can be worn and carried by a user. Preferably the user should be able to maintain his or her normal life style including, working, carrying out exercise, etc, while wearing the device 1.

There is further provided a method of analysing a body fluid comprising the steps of attaching device 1 to the skin of a user, analysing body fluid from the user, and allowing at least a part of the fluid to be absorbed by the evaporation layer 9, and allowing at least a part of the absorbed fluid to evaporate from the evaporation layer 9. The method may include the step of replacing a replaceable attachment layer 15 or replaceable evaporation layer 9.

The skilled person understands that the various embodiments of the invention may be combined whenever possible. While the invention has been described with reference to specific exemplary embodiments, the description is in general only intended to illustrate the inventive concept and should not be taken as limiting the scope of the invention. The invention is generally defined by the claims.

1. A medical device comprising an attachment surface and an evaporation layer, and at least one hollow microneedle extending from the attachment surface, the device further comprising a flow channel, an analysis unit for analysing a body fluid, and an evaporation layer, where the flow channel is arranged to transport fluid from the microneedle to the analysis unit and from the analysis unit to the evaporation layer, such that the flow channel can release fluid into the evaporation layer.

2. The medical device according to claim 1, where the device comprises a main body, and where the at least one microneedle extends from the main body, and where the analysis unit is comprised in the main body.

3. The medical device according to claim 1 where the transport of fluid through the medical device can be driven by the evaporation layer.

4. The medical device according to claim 3 where the flow is initiated by a single use pump.

5. The medical device according to claim 1, where the device comprises at least one heat-generating electronic unit and the evaporation layer is arranged in contact with the at least one heat-generating electronic unit.

6. The medical device according to claim 1, comprising a heat conducting element that is arranged between the attachment surface and the evaporation layer.

7. The medical device according to claim 6 where the heat conducting element is arranged between the evaporation layer and a heat generating electronic unit.

8. The medical device according to claim 5, where the heat-generating electronic unit or the heat conducting element is provided with at least one surface area increasing element.

9. The medical device according to claim 1, where the evaporation layer has antimicrobial activity.

10. The medical device according to claim 1, where the evaporation layer can be removed from the medical device and be replaced with another evaporation layer.

11. The medical device according to claim 1, comprising a skin attachment layer which comprises the attachment surface, where said skin attachment layer can be removed from the device and replaced with another skin attachment layer.

12. The medical device according to claim 1, where the skin attachment layer comprises the at least one microneedle.

13. An evaporation layer for attaching to a medical device according to claim 11.

14. A skin attachment layer for attaching to a medical device according to claim 10.

15. The skin attachment layer according to claim 14, said attachment layer comprising a comprising at least one microneedle.

16. A method of analysing a body fluid comprising the steps of attaching a device according to claim 1 to the skin of a user, collecting body fluid from the user with use of the at least one microneedle, using the analysis unit to analyse the body fluid, and allowing at least a part of the fluid to be absorbed by the evaporation layer, and allowing at least a part of the fluid to evaporate from the evaporation layer.
